SEQUENCE LISTING (76 sequences)

SEQ ID NO: 1 - protein, 10 aa, Artificial Sequence
  Foot protein 1 decapeptide repeat
AKPSYPPTYK

SEQ ID NO: 2 - protein, 52 aa, Artificial Sequence
  Foot protein type 3 (FP-3, Mytilus edulis)
ADYYGPKYGPPRRYGGGNYNRYGGSRRYGGYKGWNNGWKRGRWGRKYYEFEF

SEQ ID NO: 3 - protein, 75 aa, Artificial Sequence
  Foot protein type 5 (FP-5, Mytilus edulis)
SSEEYKGGYYPGNAYHYHSGGSYHGSGYHGGYKGKYYGKAKKYYYKYKNSGKYKYLKKARKYHRKGYKKYYGGSS

SEQ ID NO: 4 - protein, 76 aa, Artificial Sequence
  Foot protein type 5 (FP-5, Mytilus galloprovincialis)
SSEEYKGGYYPGNTYHYHSGGSYHGSGYHGGYKGKYYGKAKKYYYKYKNSGKYKYLKKARKYHRKGYKKYYGGGSS

SEQ ID NO: 5 - protein, 194 aa, Artificial Sequence
  Hybrid mussel adhesive protein (FP-151, MEFP-5 based Kollodis)
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 6 - protein, 196 aa, Artificial Sequence
  Hybrid mussel adhesive protein (FP-151, MGFP-5 based)
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 7 - protein, 11 aa, Artificial Sequence
  Fibronectin I-derived peptide (KYILRWRPKNS)
KYILRWRPKNS

SEQ ID NO: 8 - protein, 16 aa, Artificial Sequence
  Fibronectin III-derived peptide (YRVRVTPKEKTGPMKE)
YRVRVTPKEKTGPMKE

SEQ ID NO: 9 - protein, 9 aa, Artificial Sequence
  Fibronectin III-derived peptide (SPPRRARVT)
SPPRRARVT

SEQ ID NO: 10 - protein, 9 aa, Artificial Sequence
  Fibronectin III-derived peptide (ATETTITIS)
ATETTITIS

SEQ ID NO: 11 - protein, 32 aa, Artificial Sequence
  Fibronectin III-derived peptide (VSPPRRARVTDATETTITISWRTKTETITGFG)
VSPPRRARVTDATETTITISWRTKTETITGFG

SEQ ID NO: 12 - protein, 12 aa, Artificial Sequence
  Fibronectin III-derived peptide (ANGQTPIQRYIK)
ANGQTPIQRYIK

SEQ ID NO: 13 - protein, 11 aa, Artificial Sequence
  Fibronectin III-derived peptide (KPDVRSYTITG)
KPDVRSYTITG

SEQ ID NO: 14 - protein, 30 aa, Artificial Sequence
  Fibronectin-derived peptide (PRARITGYIIKYEKPGSPPREVVPRPRPGV)
PRARITGYIIKYEKPGSPPREVVPRPRPGV

SEQ ID NO: 15 - protein, 8 aa, Artificial Sequence
  Fibronectin III-derived peptide (WQPPRARI)
WQPPRARI

SEQ ID NO: 16 - protein, 19 aa, Artificial Sequence
  Fibronectin III-derived peptide (WQPPRARITGYIIKYEKPG)
WQPPRARITGYIIKYEKPG

SEQ ID NO: 17 - protein, 17 aa, Artificial Sequence
  Fibronectin III-derived peptide (YEKPGSPPREVVPRPRP)
YEKPGSPPREVVPRPRP

SEQ ID NO: 18 - protein, 15 aa, Artificial Sequence
  Fibronectin III-derived peptide (KNNQKSEPLIGRKKT)
KNNQKSEPLIGRKKT

SEQ ID NO: 19 - protein, 12 aa, Artificial Sequence
  Laminin alpha1-derived peptide (GLIYYVAHQNQM)
GLIYYVAHQNQM

SEQ ID NO: 20 - protein, 12 aa, Artificial Sequence
  Laminin alpha1-derived peptide (RKRLQVQLSIRT)
RKRLQVQLSIRT

SEQ ID NO: 21 - protein, 12 aa, Artificial Sequence
  Laminin alpha2-derived peptide (GLLFYMARINHA)
GLLFYMARINHA

SEQ ID NO: 22 - protein, 12 aa, Artificial Sequence
  Laminin alpha3-derived peptide (KNSFMALYLSKG)
KNSFMALYLSKG

SEQ ID NO: 23 - protein, 12 aa, Artificial Sequence
  Laminin alpha4-derived peptide (VVRDITRRGKPG)
VVRDITRRGKPG

SEQ ID NO: 24 - protein, 12 aa, Artificial Sequence
  Laminin alpha4-derived peptide (RAYFNGQSFIAS)
RAYFNGQSFIAS

SEQ ID NO: 25 - protein, 12 aa, Artificial Sequence
  Laminin alpha4-derived peptide (GEKSQFSIRLKT)
GEKSQFSIRLKT

SEQ ID NO: 26 - protein, 20 aa, Artificial Sequence
  Laminin alpha4-derived peptide (TLFLAHGRLVFMFNVGHKKL)
TLFLAHGRLVFMFNVGHKKL

SEQ ID NO: 27 - protein, 12 aa, Artificial Sequence
  Laminin alpha4-derived peptide (TLFLAHGRLVFM)
TLFLAHGRLVFM

SEQ ID NO: 28 - protein, 12 aa, Artificial Sequence
  Laminin alpha4-derived peptide (LVFMFNVGHKKL)
LVFMFNVGHKKL

SEQ ID NO: 29 - protein, 12 aa, Artificial Sequence
  Laminin alpha4-derived peptide (GAAWKIKGPIYL)
GAAWKIKGPIYL

SEQ ID NO: 30 - protein, 12 aa, Artificial Sequence
  Laminin alpha4-derived peptide (VIRDSNVVQLDV)
VIRDSNVVQLDV

SEQ ID NO: 31 - protein, 12 aa, Artificial Sequence
  Laminin alpha5-derived peptide (GKNTGDHFVLYM)
GKNTGDHFVLYM

SEQ ID NO: 32 - protein, 13 aa, Artificial Sequence
  Laminin alpha5-derived peptide (RLVSYSGVLFFLK)
RLVSYSGVLFFLK

SEQ ID NO: 33 - protein, 12 aa, Artificial Sequence
  Laminin alpha5-derived peptide (GPLPSYLQFVGI)
GPLPSYLQFVGI

SEQ ID NO: 34 - protein, 12 aa, Artificial Sequence
  Laminin alpha5-derived peptide (RNRLHLSMLVRP)
RNRLHLSMLVRP

SEQ ID NO: 35 - protein, 12 aa, Artificial Sequence
  Laminin alpha5-derived peptide (LVLFLNHGHFVA)
LVLFLNHGHFVA

SEQ ID NO: 36 - protein, 12 aa, Artificial Sequence
  Laminin alpha5-derived peptide (AGQWHRVSVRWG)
AGQWHRVSVRWG

SEQ ID NO: 37 - protein, 12 aa, Artificial Sequence
  Laminin alpha5-derived peptide (KMPYVSLELEMR)
KMPYVSLELEMR

SEQ ID NO: 38 - protein, 7 aa, Artificial Sequence
  Laminin alpha5-derived peptide (RYVVLPR)
RYVVLPR

SEQ ID NO: 39 - protein, 12 aa, Artificial Sequence
  Laminin-derived peptide (VRWGMQQIQLVV)
VRWGMQQIQLVV

SEQ ID NO: 40 - protein, 12 aa, Artificial Sequence
  Laminin-derived peptide (TVFSVDQDNMLE)
TVFSVDQDNMLE

SEQ ID NO: 41 - protein, 13 aa, Artificial Sequence
  Laminin-derived peptide (APMSGRSPSLVLK)
APMSGRSPSLVLK

SEQ ID NO: 42 - protein, 12 aa, Artificial Sequence
  Laminin-derived peptide (VLVRVERATVFS)
VLVRVERATVFS

SEQ ID NO: 43 - protein, 10 aa, Artificial Sequence
  Laminin-derived peptide (RNIAEIIKDI)
RNIAEIIKDI

SEQ ID NO: 44 - protein, 6 aa, Artificial Sequence
  Collagen I-derived peptide (KGHRGF)
KGHRGF

SEQ ID NO: 45 - protein, 12 aa, Artificial Sequence
  Collagen IV-derived peptide (TAGSCLRKFSTM)
TAGSCLRKFSTM

SEQ ID NO: 46 - protein, 13 aa, Artificial Sequence
  Collagen IV-derived peptide (GEFYFDLRLKGDK)
GEFYFDLRLKGDK

SEQ ID NO: 47 - protein, 16 aa, Artificial Sequence
  Vitronectin-derived peptide (KKQRFRHRNRKGYRSQ)
KKQRFRHRNRKGYRSQ

SEQ ID NO: 48 - protein, 4 aa, Artificial Sequence
  Bone sialoprotein-derived peptide (KRSR)
KRSR

SEQ ID NO: 49 - protein, 4 aa, Artificial Sequence
  Bone sialoprotein-derived peptide (KRRA)
KRRA

SEQ ID NO: 50 - protein, 3 aa, Artificial Sequence
  Fibronectin-derived peptide (RGD)
RGD

SEQ ID NO: 51 - protein, 4 aa, Artificial Sequence
  Fibronectin-derived peptide (RGDV)
RGDV

SEQ ID NO: 52 - protein, 12 aa, Artificial Sequence
  Fibronectin-derived peptide (PQVTRGDVFTMP)
PQVTRGDVFTMP

SEQ ID NO: 53 - protein, 6 aa, Artificial Sequence
  Fibronectin-derived peptide (GRGDSP)
GRGDSP

SEQ ID NO: 54 - protein, 20 aa, Artificial Sequence
  Fibronectin-derived peptide (PHSRNSGSGSGSGSGRGDSP)
PHSRNSGSGSGSGSGRGDSP

SEQ ID NO: 55 - protein, 7 aa, Artificial Sequence
  Fibronectin-derived peptide (EDGIHEL)
EDGIHEL

SEQ ID NO: 56 - protein, 9 aa, Artificial Sequence
  Tenascin-derived peptide (VAEIDGIEL)
VAEIDGIEL

SEQ ID NO: 57 - protein, 8 aa, Artificial Sequence
  Tenascin-derived peptide (VFDNFVLK)
VFDNFVLK

SEQ ID NO: 58 - protein, 25 aa, Artificial Sequence
  Fibronectin domain III mimetic (RGDSGSGSGSGSGANGQTPIQRYIK)
RGDSGSGSGSGSGANGQTPIQRYIK

SEQ ID NO: 59 - protein, 224 aa, Artificial Sequence
  Fibronectin domain III mimetic (ANGQTPIQRYIK - FP151 - RGD)
ANGQTPIQRYIKAKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYKRGDSGSGSGSGSGIDAPS

SEQ ID NO: 60 - protein, 20 aa, Artificial Sequence
  2 times repeated sequence derived from foot protein 1 (FP-1)
AKPSYPPTYKAKPSYPPTYK

SEQ ID NO: 61 - protein, 60 aa, Artificial Sequence
  6 times repeated sequence derived from foot protein 1 (FP-1)
AKPSYPPTYKAKPSYPPTYKAKPSYPPTYKAKPSYPPTYKAKPSYPPTYKAKPSYPPTYK

SEQ ID NO: 62 - protein, 39 aa, Artificial Sequence
  Partial sequence of foot protein type 2 (FP-2)
EVHACKPNPCKNNGRCYPDGKTGYKCKCVGGYSGPTCAC

SEQ ID NO: 63 - protein, 46 aa, Artificial Sequence
  Foot protein type 3 (FP-3, Mytilus galloprovincialis  mgfp-3A)
ADYYGPKYGPPRRYGGGNYNRYGRRYGGYKGWNNGWKRGRWGRKYY

SEQ ID NO: 64 - protein, 50 aa, Artificial Sequence
  Foot protein type 3 (FP-3, Mytilus edulis mefp-3F)
ADYYGPNYGPPRRYGGGNYNRYNGYGGGRRYGGYKGWNNGWNRGRRGKYW

SEQ ID NO: 65 - protein, 44 aa, Artificial Sequence
  Foot protein type 3 (FP-3, Mytilus californianus)
GAYKGPNYNYPWRYGGKYNGYKGYPRGYGWNKGWNKGRWGRKYY

SEQ ID NO: 66 - protein, 60 aa, Artificial Sequence
  Partial sequence from foot protein type 4 (Mytilus californianus)
GHVHRHRVLHKHVHNHRVLHKHLHKHQVLHGHVHRHQVLHKHVHNHRVLHKHLHKHQVLH

SEQ ID NO: 67 - protein, 76 aa, Artificial Sequence; identical to SEQ ID NO: 4
  Foot protein 5 (FP-5, Mytilus edulis)

SEQ ID NO: 68 - protein, 71 aa, Artificial Sequence
  Foot protein 5 (FP-5, Mytilus coruscus)
YDDYSDGYYPGSAYNYPSGSHWHGHGYKGKYYGKGKKYYYKFKRTGKYKYLKKARKYHRKGYKKHYGGSSS

SEQ ID NO: 69 - protein, 99 aa, Artificial Sequence
  Mussel adhesive protein foot protein type 6
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 70 - protein, 192 aa, Artificial Sequence
  Hybrid mussel adhesive protein (FP-151, MCFP-5 based)
AKPSYPPTYKAKPSYPPTYKAKPSYPPTYKAKPSYPPTYKAKPSYPPTYKAKPSYPPTYKYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 71 - protein, 177 aa, Artificial Sequence
  Hybrid mussel adhesive protein (FP-131)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKL

SEQ ID NO: 72 - protein, 180 aa, Artificial Sequence
  Hybrid mussel adhesive protein (FP-251)
MEVHACKPNPCKNNGRCYPDGKTGYKCKCVGGYSGPTCACSSEEYKGGYYPGNSNHYHSGGSYHGSGYHGGYKGKYYGKAKKYYYKYKNSGKYKYLKKARKYHRKGYKKYYGGSSEFEFAKPSYPPTYKAKPSYPPTYKAKPSYPPTYKAKPSYPPTYKAKPSYPPTYKAKPSYPPTYKK

SEQ ID NO: 73 - protein, 182 aa, Artificial Sequence
  Hybrid mussel adhesive protein (FP-353)
GCRADYYGPKYGPPRRYGGGNYNRYGGSRRYGGYKGWNNGWKRGRWGRKYYEFEFYDGYSDGYYPGSAYNYPSGSHGYHGHGYKGKYYGKGKKYYYKYKRTGKYKYLKKARKYHRKGYKKYYGGGSSGCRADYYGPKYGPPRRYGGGNYNRYGGSRRYGGYKGWNNGWKRGRWGRKYYEFEF

SEQ ID NO: 74 - protein, 309 aa, Artificial Sequence
  Hybrid mussel adhesive protein (FP-13151)
AKPSYPPTYKAKPSYPPTYKAKPSYPPTYKAKPSYPPTYKAKPSYPPTYKAKPSYPPTYKGCRADYYGPKYGPPRRYGGGNYNRYGGSRRYGGYKGWNNGWKRGRWGRKYYEFEFAKPSYPPTYKAKPSYPPTYKAKPSYPPTYKAKPSYPPTYKAKPSYPPTYKAKPSYPPTYKKLYDGYSDGYYPGSAYNYPSGSHGYHGHGYKGKYYGKGKKYYYKYKRTGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 75 - protein, 10 aa, Artificial Sequence
  Spacer linker peptide
SGSGSGSGSG

SEQ ID NO: 76 - protein, 12 aa, Artificial Sequence
  Laminin alpha5-derived peptide (PGRWHKVSVRWE)
PGRWHKVSVRWE

(19) United States
(12) Patent Application Publication
(10) Pub. No.: US 2023/0416340 A1
(43) Pub. Date: Dec. 28, 2023

(54) PROTEIN SUBSTRATE TO BIND GROWTH FACTOR

(71) Applicant: TME THERAPEUTICS CO., LTD., Incheon (KR)

(72) Inventors: Sang Jae LEE, Seoul (KR); Bong Jin HONG, Pohang-si, Gyeongsangbuk-do (KR); Min Chul PARK, Seongnam-si, Gyeonggi-do (KR)

(73) Assignee: TME THERAPEUTICS CO., LTD., Incheon (KR)

(21) Appl. No.: 18/253,415

(22) PCT Filed: Dec. 16, 2021
(86) PCT No.: PCT/KR2021/019159

§ 371 Date: May 18, 2023

Related U.S. Application Data
(60) Provisional application No. 63/126,245, filed on Dec. 16, 2020.

(51) Int. Cl. C07K 14/78; C07K 14/705

(52) U.S. Cl. CPC C07K 14/78; C07K 14/70546

(57) ABSTRACT

Protein substrates are provided comprising integrin binding motif and growth factor (GF) binding peptide motif that enable binding to and activation of both a growth factor receptor and an integrin, simultaneously or selectively. The substrate proteins may be in the form of a mussel adhesive protein where extracellular matrix (ECM) protein derived integrin binding peptide motifs and GF binding peptide motifs are recombinantly incorporated into the mussel adhesive protein. Also provided are use of the protein substrate for regulating cellular behavior including cell adhesion, migration, or proliferation which are essential process in bioprocess, wound healing, tissue engineering, and therapeutic application. In other embodiments, the present invention provides a method and composition of stabilizing and protecting a growth factor from protease digestion or internalization into cells (or cellular uptake) in order to maintain the persistent and durable function of growth factors.

### TECHNICAL FIELD

The present invention relates to an ECM-mimetic and growth factor complex, comprising a protein substrate having both integrin binding peptide motif and growth factor/cytokine binding peptide motif. In particular embodiments, the present invention is directly related to a protein substrate comprising one of FGF, TGFβ, PDGF, and VEGF binding peptide motifs and one of integrin αv, α2, α4, α5, and α9 binding motifs derived from fibronectin, collagen, laminin, vitronectin, and tenascin.

### BACKGROUND ART

Cell adhesion receptors, integrins, and growth factor receptors are important molecular determinants in providing specificity for signaling during development and/or pathological processes. Although integrins and growth factor receptors can independently propagate intracellular signals, the synergy of signals provided by the extracellular matrix (ECM) and growth factors (GFs) appears to regulate complex processes, including blood vessel development during embryogenesis, wound healing as well as tumor growth/metastasis.

GFs are involved in the regulation of a variety of cellular processes and typically act as signaling molecules between cells. They promote cell proliferation, differentiation and maturation, which vary in growth factors. Most growth factors act in a diffusible manner and are generally unstable in a tissue environment. This prolonged retention is considered to maintain the activity of growth factors in cells or in their environment and to be advantageous in bioprocess, regenerative medicine applications.

Thus, many attempts have been made to improve the performance of growth factors (e.g., their active period and stability). The most common strategy to prolong growth factors retention in their environment is to anchor growth factor on solid substrates by chemical bonding and those substrates could be used for many medical and biological applications including wound healing, tissue engineering, etc. (Mirhamed Hajimiri, et al, Growth factor conjugation: Strategies and applications, J. Biomedical Materials Research, Volume 103, Issue 2. 2015 p 819-838). In addition, it is very important to add biofunctionality such as the regulation of cell functions to biomaterials used for artificial organs. Modification of growth factors for immobilization on, or for high-affinity binding to cells or scaffold biomaterials has been performed by various researchers. (See Seiichi Tada, et al, Design and Synthesis of Binding Growth Factors, Int J Mol Sci. 2012; 13(5): 6053-6072). But, most of them are of limited effectiveness, mainly due to loss of growth factor activity when associated with carriers, inefficient release control of the growth factor and poor protection from proteolysis and/or degradation.

Extracellular matrix contains numerous components such as adhesive molecules, notch signaling molecules, traction-enabling adhesion molecules and proteoglycan molecules to bind to growth factors and modulate a number of their activity (Cao L., et al. 2009 Promoting angiogenesis via manipulation of VEGF responsiveness with notch signaling. Biomaterials 30, 4085-4093; Discher D. E., et al. 2005 Tissue cells feel and respond to the stiffness of their substrate. Science 310, 1139-1143; Ramirez F.& Rifkin D. B., 2003 Cell signaling events: a view from the matrix. Matrix Biol. 22, 101-107).

Many ECM proteins have binding sites for both growth factors and cell adhesion which allow growth factors to be released locally and bind to their cell surface receptors. Thus, the ECM functions as a cofactor and presents the growth factor for cell surface receptors. Further, localization of growth factors by ECM binding contributes to the establishment of gradients of soluble chemokines and growth factor morphogens, which play an essential role in developmental processes. Growth factors can also be sequestered to the ECM, which hereby function as a localized reservoir. Degradation of ECM will then release the solid inactive growth factors that are transformed to active soluble ligands (Kim, S.-H., et al. 2011. Extracellular matrix and cell signalling: the dynamic cooperation of integrin, proteoglycan and growth factor receptor. The Journal of endocrinology, 209(2), p 139-51; Hynes, et al. 2012. Overview of the matrisome—an inventory of extracellular matrix constituents and functions. Cold Spring Harbor perspectives Hynes, R. O., 2009; The extracellular matrix: not just pretty fibrils. Science (New York, N.Y.), 326(5957), pp. 1216-9). Some growth factors are known to act in a non-diffusible manner and such growth factors are HB-EGF, TGF, TNF, and CSF while most growth factors act in a diffusible manner. (Seiichi Tada, et al. Int J Mol Sci. 2012; 13(5): 6053-6072. Design and Synthesis of Binding Growth Factors).

The purpose of the present invention is to provides more simple and reliable protein substrate to immobilize grow factor with long-lasting stability and functionality by utilizing ECM-derived GF binding peptide motif as well as integrin binding motif.

### DISCLOSURE OF INVENTION

### Technical Problem

An object of the present invention is to provide a protein substrate comprising a recombinant adhesive protein genetically functionalized with an integrin binding motif and a heparin binding motif which is capable of binding or sequestering growth factors.

Another object of the present invention is to provide an extracellular microenvironment surface to regulate cell plasticity, wherein said microenvironment surface comprises the protein substrate of any one of claims 1 to 17 that can induce combinatorial signaling via activating simultaneously integrins and growth factor receptors.

### Solution to Problem

To achieve the objects, in an aspect, the present invention provides a protein substrate comprising a recombinant adhesive protein genetically functionalized with an integrin binding motif and a heparin binding motif which is capable of binding or sequestering growth factors.

In an embodiment of the present invention, said heparin binding motif can be derived from fibronectin domain III, laminin globular domain, heparin binding domain of collagen, vitronectin, or bone sialoprotein.

In a preferred embodiment of the present invention, said heparin binding motif derived from fibronectin domain III can be a peptide of KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), or KNNQKSEPLIGRKKT (SEQ ID NO: 18). In another preferred embodiment of the present invention, said heparin binding motif derived from laminin globular domain can be a peptide of GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), or RNIAEIIKDI (SEQ ID NO: 43). In another preferred embodiment of the present invention, said heparin binding motif derived from heparin binding domain of collagen can be a peptide of KGHRGF (SEQ ID NO: 44), TAGSCLRKFSTM (SEQ ID NO: 45), or GEFYFDLRLKGDK (SEQ ID NO: 46). In another preferred embodiment of the present invention, said heparin binding motif derived from heparin binding domain of vitronectin can be a peptide of KKQRFRHRNRKGYRSQ (SEQ ID NO: 47). In another preferred embodiment of the present invention, said heparin binding motif derived from heparin binding domain of bone sialoprotein can be a peptide of KRSR (SEQ ID NO: 48), or KRRA (SEQ ID NO: 49).

In an embodiment of the present invention, said heparin binding motif can be capable of binding basic fibroblast growth factor (bFGF), transforming growth factor β (TGF-β), or platelet derived growth factor (PDGF).

In another embodiment of the present invention, said integrin binding motif can be αvβ3-, αvβ6-, αvβ8-, α5β1-, or α9β1 binding peptide. In a preferred embodiment of the present invention, said integrin binding motif can be capable of activating integrin αvβ6 and said heparin binding motif can be capable of binding TGF-β. In another preferred embodiment of the present invention, said integrin binding motif can be capable of activating integrin α5β1 or α9β1 and said heparin binding motif can be capable of binding bFGF.

In an embodiment of the present invention, the recombinant adhesive protein can be derived from a recombinant mussel adhesive protein. In a preferred embodiment of the present invention, the recombinant mussel adhesive protein may comprises, consists essentially of, or consists of the peptide sequence of SEQ ID NOs: 1-6, and 60-74. In another preferred embodiment of the present invention, the integrin binding motif and/or the heparin binding motif can be bound to N-terminal and/or C-terminal of the recombinant adhesive protein. In another preferred embodiment of the present invention, both of the integrin binding motif and the heparin binding motif can be bound to N-terminal or C-terminal of the recombinant adhesive protein.

In an embodiment of the present invention, the integrin binding motif and the heparin binding motif can be connected via a spacer linker peptide. In a preferred embodiment of the present invention, the spacer linker peptide can be a peptide of SEQ ID NO: 75.

In another aspect, the present invention provides an extracellular microenvironment surface to regulate cell plasticity.

In an embodiment of the present invention, said microenvironment surface can comprise the protein substrate of the present invention that can induce combinatorial signaling via activating simultaneously integrins and growth factor receptors. In a preferred embodiment of the present invention, said cell plasticity can be epithelial-mesenchymal transition.

Protein substrates are provided in the form of recombinant adhesive protein comprising GF binding peptide motif and integrin binding peptide motif. The protein substrates induce or manipulate a broad range of cellular behaviors including cell adhesion, migration, growth, and survival by activating growth factor receptor or integrin, simultaneously or sequentially.

In one aspect, the present invention provides a protein substrate in the form of recombinant adhesive protein comprising three domains of
1) A mussel adhesive protein domain that adheres to the surface of cells, tissues, or any substrate such as plastics and glass;
2) A growth factor binding domain which is capable of immobilizing or sequester growth factors to activate or inhibit a cognate growth factor receptor; and
3) An integrin binding domain which is capable of activating integrin.

According to the present invention, any recombinant mussel adhesive protein can be used for the purpose of this invention. In an embodiment of the present invention, the recombinant mussel adhesive protein may comprises, consists essentially of, or consists of the peptide of SEQ ID NOs: 1-6, and 60-74. In a preferred embodiment of the present invention, the recombinant mussel adhesive protein may be selected from foot protein 1 decapeptide repeat (SEQ ID NO: 1), foot protein 3 (SEQ ID NO: 2), foot protein 5 (SEQ ID NO: 3-4) or its combination. Preferably, the hybrid of foot protein 1 decapeptide repeat and foot protein 3, foot protein 1 decapeptide repeat and foot protein 5, or foot protein 1, foot protein 3 and foot protein 5. Preferably, the hybrid protein (SEQ ID NO: 5 and SEQ ID NO: 6) consisted of six repeats of foot protein 1 decapeptide at both the N- and C-termini of M. edulis foot protein 5 (SEQ ID NO: 3) or M. galloprovincialis foot protein 5 (SEQ ID NO: 4) is used for the present invention.

The GF binding domain in the present invention may be heparin binding or syndecan binding peptide motif derived from ECM protein including collagen, fibronectin, laminin, vitronectin, fibrinogen, tenascin, or bone sialoprotein. The growth factor bound or sequestered by heparin binding or syndecan binding motifs includes basic fibroblast growth factor (bFGF), platelet-derived growth factor (PDGF), epidermal growth factor (EGF), and vascular endothelial growth factor (VEGF), and the cytokine bound or sequestered by heparin or syndecan binding motifs are transforming growth factor β (TGF-β), interleukin-2, and interleukin-6.

In another aspect, the present invention provides a protein substrate to create synthetic extracellular microenvironment for culturing valuable cells, comprising an ECM-derived integrin binding peptides and one or more GF binding motifs to immobilize exogenous growth factors. The exogenous growth factors that are bound to heparin or syndecan binding motifs are retained within the protein substrate according to the invention.

In another aspect, the present invention provides a protein substrate for sustained growth factor delivery for bioprocess or tissue engineering application.

In another aspect, the invention provides a composition comprising the protein substrate of the first aspect and a pharmaceutically-acceptable carrier for cell therapy, wound healing or tissue engineering.

In another aspect, the present invention provides a protein substrate as a synthetic extracellular matrix retaining growth factors or cytokines.

In another aspect, the present invention provides a method of promoting cell migration including the step of using a protein substrate to bind both a growth factor receptor and an integrin.

The present invention provides a substrate to immobilize growth factors or cytokines to deliver to cells, tissues, or organs. Embodiments as well as features and advantages of the present invention will be apparent from the further descriptions herein.

### Advantageous Effects of Invention

The protein substrate of the present invention may be used for cell culture related applications, for example, surface coating for bioprocess for stem cell expansion, delivery of growth factor for tissue engineering, or therapeutic applications.

### BRIEF DESCRIPTION OF DRAWINGS

FIG. 1a represents two basic formulas of a protein substrate presenting integrin binding motif and heparin binding motifs which is capable of activating or binding to various growth factors. Formula A is the protein substrate having integrin binding & GF binding peptide motif at C-terminus and N-terminus, and both binding peptide motifs are incorporated at C-terminus of the protein substrate in Formula B.

FIG. 1b represents the action mechanism of the protein substrate of the present invention. For specificity, the protein substrate coated surface forms island like topography that allows specific interaction between binding motifs and growth factor receptor or integrin as represented in FIG. 1b.

FIG. 2a and FIG. 2b represent layouts of array of heparin binding peptide motif to screen any peptide motif having high affinity to GF. FIG. 2a is the layout of fibronectin, collagen, and laminin derived heparin or syndecan binding peptide motif and FIG. 2b is the layout of laminin LG domain derived heparin or syndecan binding peptide.

FIG. 3 represents the calculated GF binding affinity to GF binding motif.

FIG. 4a and FIG. 4b represent the screening results of various ECM-derived GF binding peptide motif to bFGF (FIG. 4a) and TGF-β (FIG. 4b), respectively. FIG. 4c represents the screening results of laminin-derived GF binding peptide motif to bFGF, TGF-β, and PDGF, respectively.

FIG. 5a and FIG. 5b represent the sustained release of TGF-β bound to the protein substrate having high affinity to TGF-β. FIG. 5a is the layout of TGF-β binding peptide motif with high affinity to TGF-β, and FIG. 5b represents the absorbance profile of the GF binding motif with high affinity for TGF-β showing long-term sustained release of TGF-β.

FIG. 6a represents the layout ECM derived peptide motif binding to epithelial-mesenchymal transition (EMT) inducible integrin αv, α2, and α9, and FIG. 6b represents western blot results of MCF-10A, a breast epithelial cell, cultured on the EMT-inducible integrin binding motif coated surface. In FIG. 6b, the fibronectin expression is represented as an EMT marker induced TGF-β bound to the protein substrate. A protein substrate having GF binding motif (YEK & ANG0 with high affinity for TGF-β induced high expression of fibronectin while a protein substrate having GF binding motif (WQ) with low affinity for TGF-β did not induce fibronectin expression.

FIG. 7 represents the trypsin-mediated TGF-β cleavage analyzed by Western blot. TGF-β bound to protein substrate had low levels of trypsin digestion while TGF-β without protein substrate was most digested when treated with trypsin.

FIG. 8a and FIG. 8b represent the effect of GF bound to protein substrate on the growth and proliferation of human foreskin fibroblast in low serum condition (0.5% FBS). FIG. 8a is the effect of PDGF bound to protein substrate, and FIG. 8b is the effect of FGF2 bound to protein substrate on cell growth and proliferation.

### MODE FOR THE INVENTION

The present invention is directed to a protein substrate that induces signaling mediated by integrins and growth factor/cytokine receptors, simultaneously or selectively, to regulate cellular behavior. A protein substrate may be provided in the form of recombinant adhesive protein comprising three domains of
1) A recombinant mussel adhesive protein domain that adheres to the surface of cells, tissues, or any substrate such as plastics or glass;
2) A growth factor binding domain which is capable of immobilizing or sequestering growth factors or cytokines to activate or inhibit a cognate growth factor receptor or cytokine receptor; and
3) An integrin binding domain which is capable of activating integrin receptors.

As used herein, the term “a substrate” means a substance to which another substance is applied. In biology, the surface on which an organism such as a plant, fungus, or animal lives can be called as a substrate. This surface can include all biotic, or abiotic components.

As used herein, a recombinant mussel adhesive protein refers to a fusion protein comprising mussel foot protein FP-5 and mussel foot protein FP-1 decapeptide. In one embodiment, a protein substrate provided here comprise a mussel foot protein that is selected from the group consisting SEQ ID NOs: 5-6.

As used herein, the term “GF binding peptide motif” refers to a short peptide derived from heparin binding or syndecan binding domain of extracellular matrix proteins such as, including but not limited to, fibronectin domain III, laminin LG domain, collagen heparin binding domain, vitronectin heparin binding domain, or fibrinogen. In one embodiment, the GF binding peptide motif comprises 5-40 amino acids or its combination thereof. In various embodiments, the GF binding peptide motif comprises an amino acid sequence selected from the heparin binding or syndecan binding peptide group consisting of fibronectin-derived KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), KNNQKSEPLIGRKKT (SEQ ID NO: 18), or laminin-derived GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), PGRWHKVSVRWE (SEQ ID NO: 76) or collagen-derived KGHRGF (SEQ ID NO: 44), TAGSCLRKFSTM (SEQ ID NO: 45), GEFYFDLRLKGDK (SEQ ID NO: 46), or vitronectin-derived KKQRFRHRNRKGYRSQ (SEQ ID NO: 47), or bone sialoprotein derived KRSR (SEQ ID NO: 48), KRRA (SEQ ID NO: 49).

As used herein, the term “integrin binding motif” refers to a short peptide derived from extracellular matrix proteins such as, including but not limited to, fibronectin, laminin, collagen, vitronectin, or tenascin. The integrin binding motifs bind to and activate integrin αv, α5, α8 or α9 to support cell adhesion and may induce morphogenesis together with growth factor bound to the heparin binding motif. In various embodiments, the heparin binding peptide comprises an amino acid sequence selected from the group consisting of integrin av binding RGD (SEQ ID NO: 50), RGDV (SEQ ID NO: 51), PQVTRGDVFTMP (SEQ ID NO: 52), or integrin α5 binding GRGDSP (SEQ ID NO: 53), PHSRNSGSGSGSGSGRGDSP (SEQ ID NO: 54), or integrin α9 binding EDGIHEL (SEQ ID NO: 55), VAEIDGIEL (SEQ ID NO: 56), or integrin α8 binding VFDNFVLK (SEQ ID NO: 57).

In one embodiment, the present invention discloses a protein substrate that provides a GF binding peptide motif to sequester or bind to growth factors, simultaneously or selectively. Any suitable GF binding peptide motif can be selected from the group consisting heparin binding or syndecan binding motif derived from fibronectin domain III, laminin LG domain, collagen heparin domain, vitronectin heparin domain, or bone sialoprotein as described in details in the definition term of heparin binding motif above.

In one embodiment, a protein substrate to sequester or bind to basic fibroblast growth factor (bFGF) is disclosed. Generally, the GF binding peptide motif can be selected from heparin/syndecan binding domain of fibronectin, laminin and collagen for bFGF binding. Preferably, the fibronectin-derived peptide PRARITGYIIKYEKPGSPPREVVPRPRPGV (SEQ ID NO: 14), WQPPRARI (SEQ ID NO: 15), laminin-derived peptide RYVVLPR (SEQ ID NO: 38), VRWGMQQIQLVV (SEQ ID NO: 39), VLVRVERATVFS (SEQ ID NO: 42), or collagen-derived KGHRGF (SEQ ID NO: 44) can be selected to sequester or bind to bFGF.

In another embodiment, the present invention discloses a protein substrate to provide GF binding peptide motif to sequester or bind to transforming growth factor β (TGF-β). The GF binding peptide motif can be selected from heparin/syndecan binding domain of fibronectin, laminin, collagen, vitronectin, or bone sialoprotein for TGF-β binding. Preferably, fibronectin-derived motif ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), or laminin-derived peptide GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), or collagen-derived KGHRGF (SEQ ID NO: 44), TAGSCLRKFSTM (SEQ ID NO: 45), GEFYFDLRLKGDK (SEQ ID NO: 46), or vitronectin-derived KKQRFRHRNRKGYRSQ (SEQ ID NO: 47), or bone sialoprotein derived KRSR (SEQ ID NO: 48), KRRA (SEQ ID NO: 49) can be selected to bind to or sequester TGF-0. More preferably, the heparin binding motif can be selected from fibronectin derived ANGQTPIQRYIK (SEQ ID NO: 12), KPDVRSYTITG (SEQ ID NO: 13), YEKPGSPPREVVPRPRP (SEQ ID NO: 17), or laminin derived KNSFMALYLSKG (SEQ ID NO: 22), RYVVLPR (SEQ ID NO: 38), GKNTGDHFVLYM (SEQ ID NO: 31), RLVSYSGVLFFLK (SEQ ID NO: 32), VLVRVERATVFS (SEQ ID NO: 42), or vitronectin derived KKQRFRHRNRKGYRSQ (SEQ ID NO: 47), or bone sialoprotein derived KRSR (SEQ ID NO: 48), KRRA (SEQ ID NO: 49).

In another embodiment, the present invention discloses a protein substrate to provide GF binding peptide motif to sequester or bind to platelet-derived growth factor (PDGF). The PDGF binding motif can be selected from laminin derived motif GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), RNIAEIIKDI (SEQ ID NO: 43), PGRWHKVSVRWE (SEQ ID NO: 76), or vitronectin derived KKQRFRHRNRKGYRSQ (SEQ ID NO: 47), or bone sialoprotein derived KRSR (SEQ ID NO: 48), KRRA (SEQ ID NO: 49). More preferably, the PDGF binding motif can be selected from RKRLQVQLSIRT (SEQ ID NO: 20), KNSFMALYLSKG (SEQ ID NO: 22), RYVVLPR (SEQ ID NO: 38), GKNTGDHFVLYM (SEQ ID NO: 31), VLVRVERATVFS (SEQ ID NO: 42), or vitronectin derived KKQRFRHRNRKGYRSQ (SEQ ID NO: 47), or bone sialoprotein derived KRSR (SEQ ID NO: 48).

The present invention further discloses a protein substrate for sustained release of growth factor in physiological conditions.

It is well known that interactions with heparin sulfate occurring in the extracellular matrix have been shown directly to regulate the diffusion of growth factors such as FGF (Duchesne L, et al, Transport of fibroblast growth factor 2 in the pericellular matrix is controlled by the spatial distribution of its binding sites in heparan sulfate. PLoS Biol. 2012; 10(7):e1001361., Dowd C J, et al, Heparan sulfate mediates bFGF transport through basement membrane by diffusion with rapid reversible binding. J Biol Chem. 1999 19; 274(8):5236-44) as well as the storage and release of FGFs in tissue homeostasis (Bashkin P, et al., asic fibroblast growth factor binds to subendothelial extracellular matrix and is released by heparitinase and heparin-like molecules. Biochemistry. 1989 21; 28(4):1737-43).

In one embodiment, the present invention provides a protein substrate as sustained release system of TGF-β without functional loss over a period of days in physiological conditions. The protein substrate provides a TGF-β binding motif, selected from KNSFMALYLSKG (SEQ ID NO: 22), RYVVLPR (SEQ ID NO: 38), GKNTGDHFVLYM (SEQ ID NO: 31), RLVSYSGVLFFLK (SEQ ID NO: 32), VLVRVERATVFS (SEQ ID NO: 42), for sustained release of TGF-β in physiological conditions.

The present invention also discloses a protein substrate to provide integrin binding peptide motif and GF binding peptide motif at the same time. An integrin binding motif can be incorporated into N-terminus of the recombinant adhesive protein and a growth factor or cytokine binding motif can be incorporated into C-terminus of said adhesive protein, or vice versa.

Crosstalk between integrins and growth factor receptors has been well known. For example, Jang reported that FGF2-FNIII9-10 fusion protein exhibited a significant increase of cell adhesion and proliferation of MG63 cells compared with FNIII9-10 alone. (Jun-Hyeog Jang & Chong-Pyoung Chung, Engineering and expression of a recombinant fusion protein possessing fibroblast growth factor-2 and fibronectin fragment. Biotechnology Letters volume 26, p 1837-1840(2004)), and FNIII9-10-mediated adhesion promotes the effect of FGF1 on neurite outgrowth of PC12 cells (Choung P H, et al., Synergistic activity of fibronectin and fibroblast growth factor receptors on neuronal adhesion and neurite extension through extracellular signal-regulated kinase pathway. Biochem Biophys Res Commun, 2002, 295: 898-902).

In one embodiment, the present invention discloses a protein substrate that mimic fibronectin domain III having RGDSGSGSGSGSGANGQTPIQRYIK (SEQ ID NO: 58).

In another embodiment, the present invention discloses a protein substrate that mimic fibronectin domain III (SEQ ID NO: 59), where integrin binding motif RGD (SEQ ID NO: 50) was incorporated in its C-terminus of adhesive protein and growth factor or cytokine, for example, TGF-β binding motif ANGQTPIQRYIK (SEQ ID NO: 12) in its N-terminus of adhesive protein.

The present invention also provides a microenvironment surface that simultaneously activates integrin and growth factor receptor in order to control phenotype plasticity, which is directly related to wound healing, angiogenesis or pathogenesis such as fibrosis or tumor metastasis.

In one embodiment, the present invention provides a synthetic tumor microenvironment surface comprising said protein substrate to induce epithelial to mesenchymal transition (EMT) in epithelial cells.

EMT is a process where epithelial cells lose epithelial proteins including E-Cadherin, and gains mesenchymal markers such as N-Cadherin, Vimentin and Fibronectin. EMT is associated with many processes, including embryonic or cancer development and/or progress (Kalluri R, et al. The basics of epithelial-mesenchymal transition. J Clin Invest. 2009; 119:1420-8. Jordan N V, et al. Tracking the intermediate stages of epithelial-mesenchymal transition in epithelial stem cells and cancer. Cell Cycle. 201; 10:2865-73), wound healing and tissue repair, and cell migration. (Hosaka K, et al. Pericyte-fibroblast transition promotes tumor growth and metastasis. Proc Natl Acad Sci USA. 2016; 113:E5618-27. Yang X, et al. Silencing Snail suppresses tumor cell proliferation and invasion by reversing epithelial-to-mesenchymal transition and arresting G2/M phase in non-small cell lung cancer. Int J Oncol. 2017; 50:1251-60). While EMT events are essential for development and wound repair, it has also been recognized as a contributing factor to fibrotic diseases and cancer. Many soluble and insoluble factors including TGF-13 and ECM proteins determine the degree and duration of EMT events. Specifically, cytokines such as TGF-β, TNFα, and IL6 and hypoxia are capable of inducing EMT in various tumors.

Several extracellular matrix (ECM) proteins, including collagen-I, fibronectin, and hyaluronan, and ECM remodeling via extracellular lysyl oxidase are also implicated in regulating EMT (Hae-Yun Jung, et al. Clin Cancer Res; 21(5) Mar. 1, 2015. Molecular Pathways: Linking Tumor Microenvironment to Epithelial-Mesenchymal Transition in Metastasis). Several integrins have been known to mediate EMT. For example, EMT in regulated by integrin αvβ6 via activation of TGF-β1-Smad2/3 signaling pathway. (Wang J, et al. (2015) Interleukin-1beta promotes epithelial-derived alveolar elastogenesis via αvβ6 integrin-dependent TGF-beta activation. Cell Physiol Biochem 36:2198-2216). The expression of several integrin complexes is also unregulated during EMT, including α5β1, which binds to fibronectin, and the integrins at α1β1 & α2β1, which interact with collagen I and have been shown to mediate the disruption of E-cadherin complexes. Cellular interactions with the ECM have been shown to be modulated by ECM-associated proteins such as SPARC that is a glycoprotein to promote the interaction of collagen and α2β1.

In one embodiment, the present invention provides a protein substrate comprising integrin binding motif selected from integrin α5β1, α9β1, or αvβ3 binding motif, and cytokine binding motif selected from ANGQTPIQRYIK (SEQ ID NO: 12), KPDVRSYTITG (SEQ ID NO: 13) or YEKPGSPPREVVPRPRP (SEQ ID NO: 17) in order to induce EMT in breast cancer cell line, MCF-10A.

The present invention also provides a method to stabilize growth factors or cytokines against loss of biological activity for long term in cell culture conditions by admixing a protein substrate comprising a growth factor or cytokine binding motif with growth factor or cytokine in cell culture medium or buffer solution such as PBS buffer.

In one embodiment, a composition comprising a protein substrate presenting cytokine binding motif ANGQTPIQRYIK (SEQ ID NO: 12) and growth factor binding motif YEKPGSPPREVVPRPRP (SEQ ID NO: 17) dissolved in cell culture medium such as DMEM or buffer solution such as PBS. This composition may be stored for at least one week without loss of its biological activity as demonstrated in EMT promotion test in breast epithelial cell line, MCF-10A.

Hereinafter, the present invention will be described in detail with reference to Preparation Examples, Examples, and Experimental Examples thereof. However, it should be understood that the following Preparation Examples, Examples,

and Experimental Examples are given for the purpose of illustration of the present invention only, and are not intended to limit the scope of the present invention.

### EXAMPLES

### Example 1. Preparation of a Protein Substrate Presenting Integrin Binding Motif and Heparin Binding Motif

E. coli based protein expression system was commercialized to produce a variety of mussel adhesive proteins including fusion protein of mussel foot protein 1 and foot protein 5 in an efficient way (see US2020/0062809A1 and WO2011/115420A2), and the mussel adhesive proteins are commercially available under Trademarks MAPTrix™ marketed by Kollodis BioSciences, Inc. The method for preparation of mussel adhesive proteins are fully described in US20200/062809A1 and WO2011/115420A2 which is hereby incorporated by reference for all purposes as if fully set forth herein.

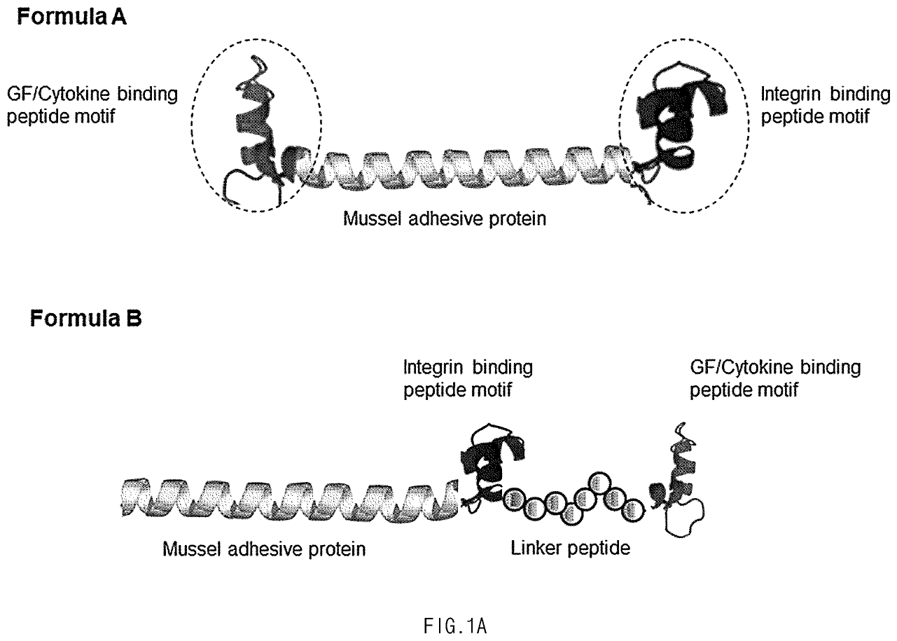

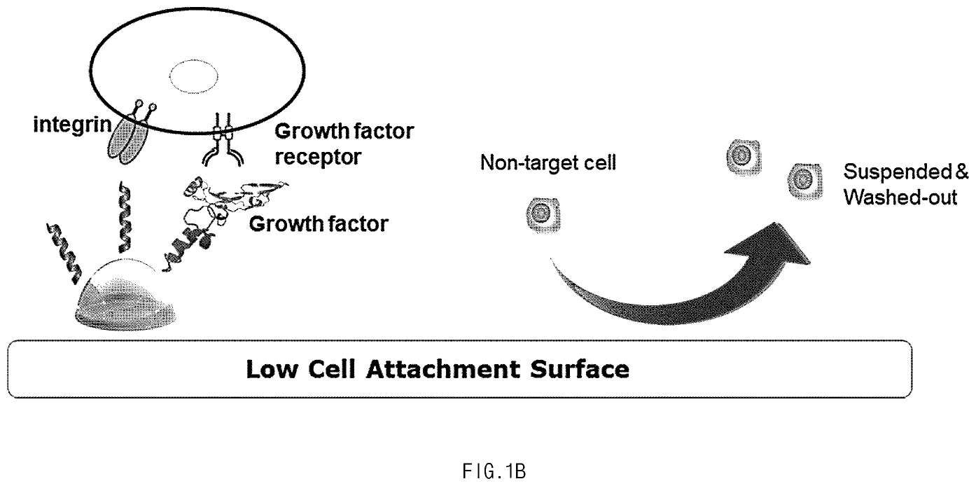

The basic formula of a protein substrate is illustrated in FIG. 1a. Two peptide motifs can be incorporated into N-terminus and C-terminus of mussel adhesive protein (Formula A), respectively as seen in FIG. 1a. Alternatively, both motifs can be incorporated into C-terminus or N-terminus (Formula B) as seen in FIG. 1a, wherein a spacer linker peptide such as SGSGSGSGSG effectively separate two peptides for synergistic effect.

Two types of protein substrates having SEQ ID NO: 58 (hereafter MAP-RGD-GF) and SEQ ID NO: 59 (hereafter GF-MAP-RGD) were recombinantly designed and expressed in E. coli expression system and purified as set forth in US2020/0062809A1 and WO2011/115420A2. A number of protein substrate having a GF binding motif was produced with the same procedure. All protein substrate was lyophilized and stored at refrigerator for further experiment.

### Example 2. Growth Factor Binding Assay

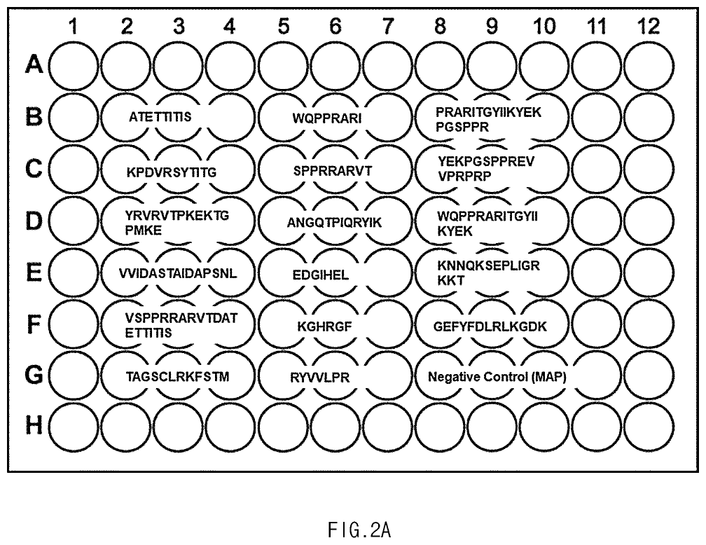

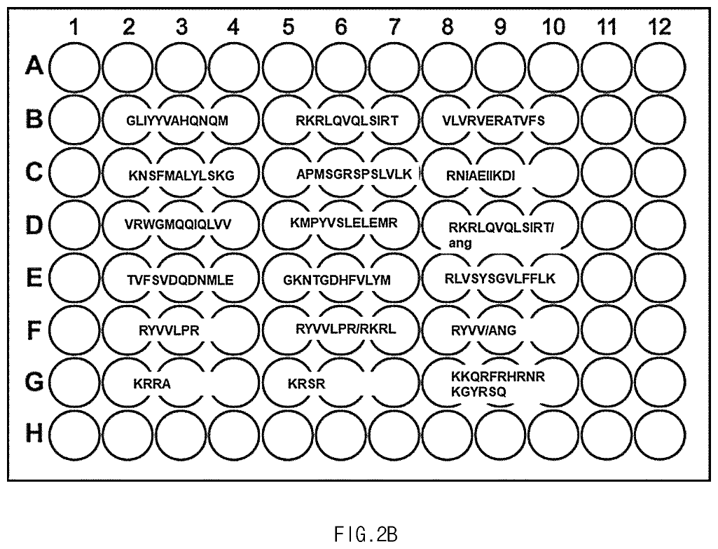

Multiple arrays for growth factor or cytokine binding assay are composed of a large number of heparin binding peptide motifs arranged in 96 well format as represented in FIG. 2a and FIG. 2b. FIG. 2a is the array layout of fibronectin, collagen, and laminin derived GF binding motif and FIG. 2b is the array layout of laminin globular domain derived GF binding motif.

For specific binding of peptide motif to a growth factor or cytokine, low cell-attachment or ultrahydrophobic surface was coated with the substrate protein wherein the substrate protein was formed as particles. So, the surface looks like particle island as illustrated in FIG. 1b. The coating method is state-of-the-art technology and detailed procedure is described in the United States patent application U.S. Ser. No. 16/546,966 developed by present inventors and hereby fully incorporated as reference. This surface can allow biomolecules such as GF or target cells to specifically bind to the peptide motif presented on the surface. Non-target biomolecules or cells are forced to be suspended, and eventually washed out.

Recombinant basic FGF, TGF-β, PDGF-BB were purchased from R&D Systems (Camarillo, CA) and Ultralow cell-attachment 96 well plate from Corning (Corning, NY). Fatty acid-free bovine serum albumin (BSA) was purchased from Sigma-Aldrich Corp. (St. Louise, MO) and fetal bovine serum from Thermo Fisher Scientific (Waltham, MA).

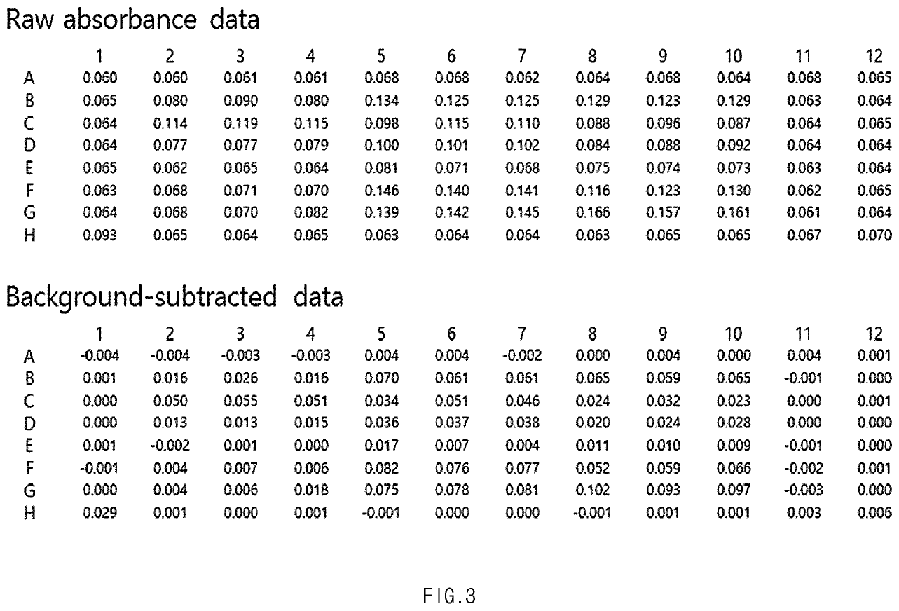

To eliminate or minimize any non-specific binding of recombinant growth factors, the 96 well plate surface coated with the substrate protein was blocked with 2 wt % BSA in PBST buffer (4 mM phosphate and 155 mM sodium chloride, 0.05 wt % Tween-20, pH 7.4) by rocking the plate at RT for 1 h. Individual recombinant growth factors (50 Nm) dissolved in PBST were then added on to the blocked well plate including the heparin binding motif and the plate was rocked at 4° C. overnight. The binding of the growth factor to the protein substrate coated on the well plate was confirmed by immunoaffinity assay. In brief, the well plate was sequentially treated with a primary antibody for the growth factor and its secondary antibody labeled with horseradish peroxidase (HRP). Finally, TMB (3,3′,5,5′-tetramethylbenzidine) substrate was chemically changed by HRP and its absorbance values at 450 nm were used to analyze the binding of the growth factors on to each binding motifs. The FIG. 3 shows TMB absorbance values corresponding to the degree of growth factor binding. The values were subtracted by the absorbance value of a blank sample without growth factors. An absorbance reading over 0.1 was considered as a significant interaction.

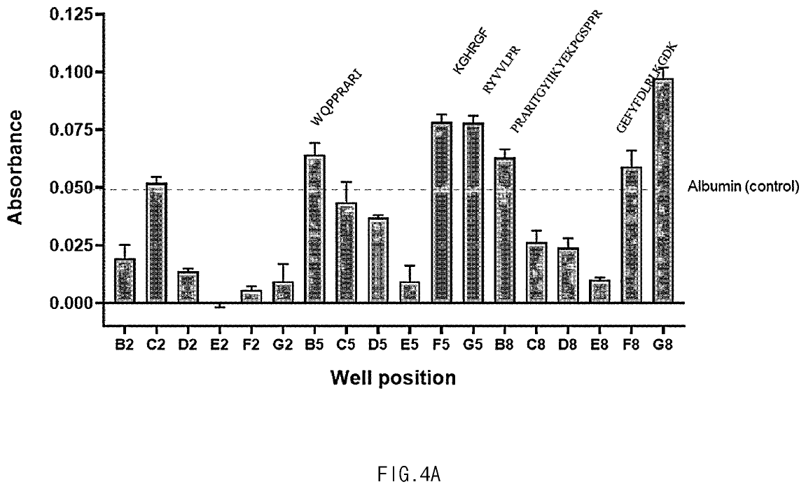

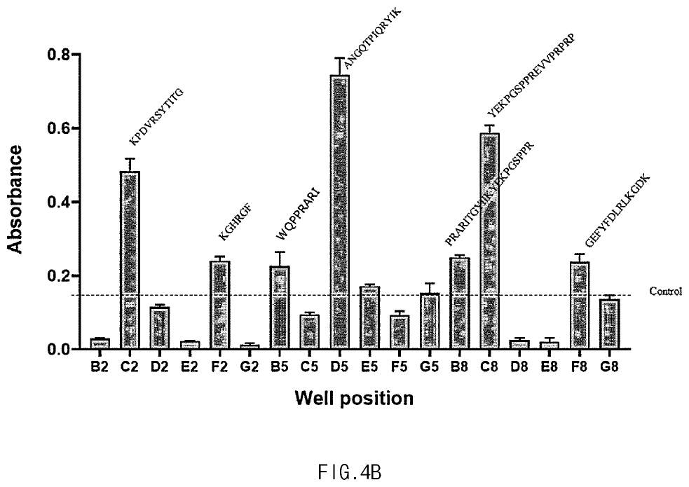

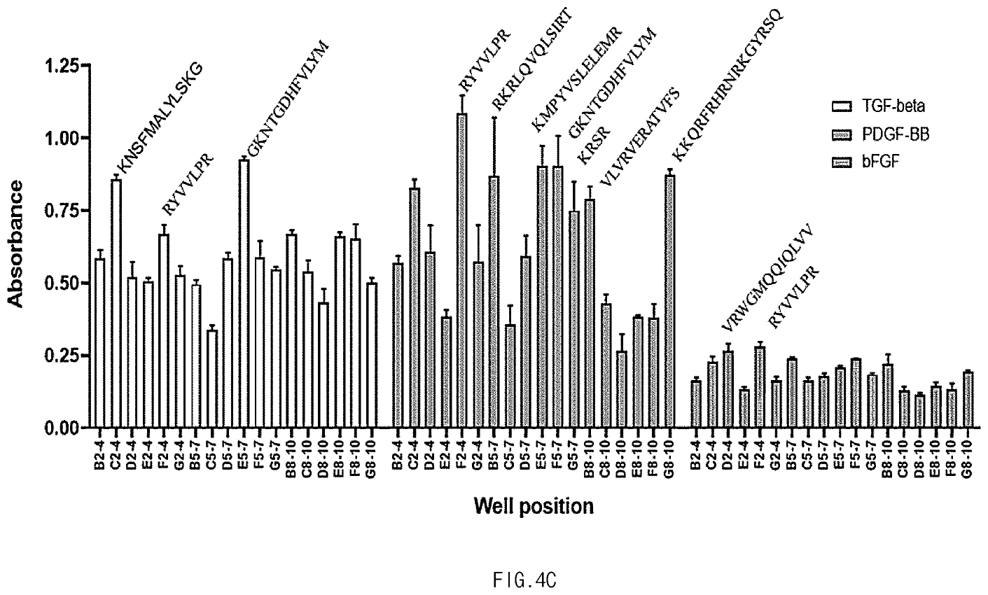

We identified fibronectin derived GF binding motifs specifically bound to bFGF, TGF-β, PDGF-BB as shown in FIG. 4a and FIG. 4b. The GF binding motif having high affinity to each growth factor are summarized in the following Table 1.

TABLE 1

| Peptide motif | Basic FGF | TGF-β | PDGF |
|---|---|---|---|
| KYILRWRPKNS | | | High |
| YRVRVTPKEKTGPMKE | | | |
| SPPRRARVT | | | |
| ATETTITIS | | | |
| VSPPRRARVTDATETTITIS- | | | |
| WRTKTETITGFG | | | |
| ANGQTPIQRYIK | High | High | |
| KPDVRSYTITG | High | | |
| PRARITGYIIKYEKPGSPPR- | | High | |
| EVVPRPRPGV | | | |
| WQPPRARI | Moderate | Moderate | |
| WQPPRARITGYIIKYEKPG | | | |
| YEKPGSPPREVVPRPRP | High | Moderate | |
| KNNQKSEPLIGRKKT | | | |
| RYVVLPR | | High | |
| KGHRGF | | | |

Most laminin derived GF binding peptide motifs showed higher affinity to TGF-β and PDGF, but relatively low affinity to bFGF, similar to that of fibronectin GF binding motif as seen in FIG. 4c.

### Example 3. Sustained Release of Growth Factor Bound to the Protein Substrate

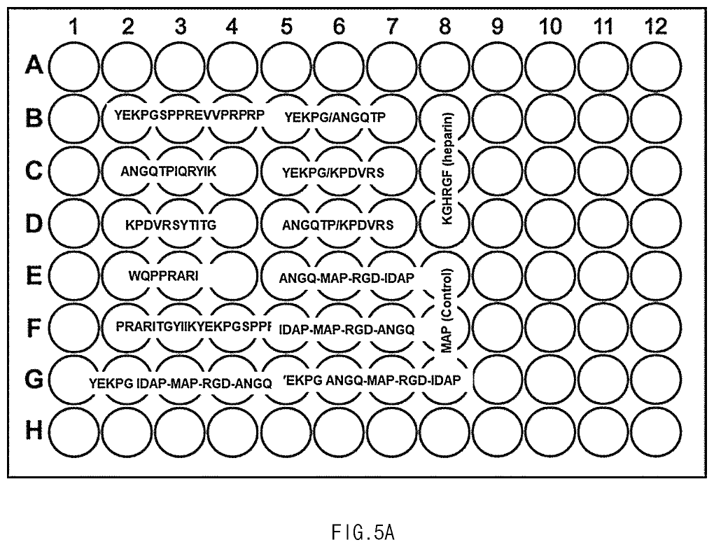

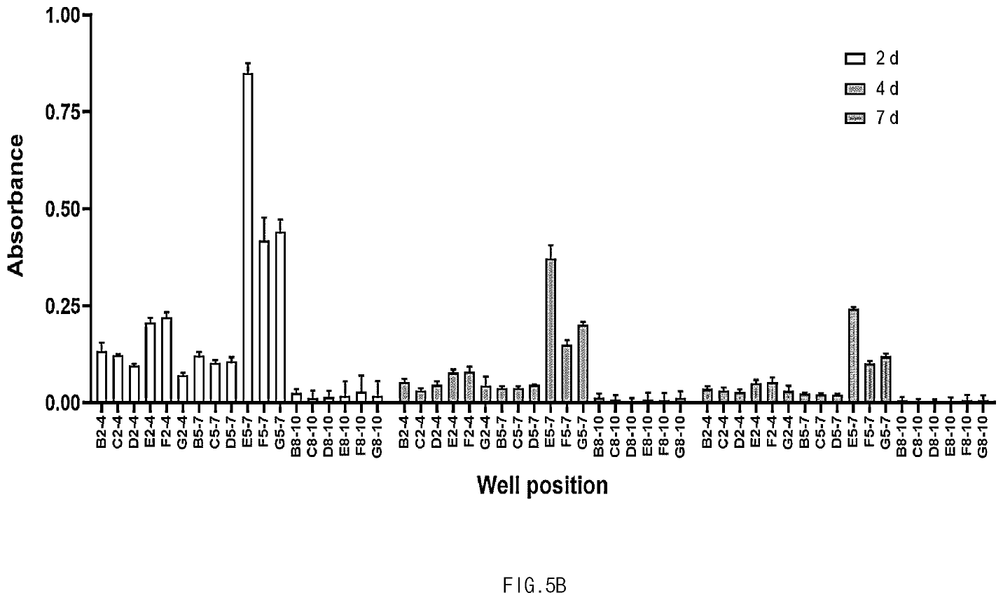

Heparin binding domain in ECM proteins has been shown to stabilize growth in physiological conditions. To determine if the GF binding peptide motif confers any protective effect on the growth factors, TGF-β was conditioned with PBS buffer or cell culture medium, DMEM, in the presence or absence of GF binding peptide motif identified in the EXAMPLE 2. bFGF (0.5 jig) was added to the protein substrate coated surface and incubated at 37° C. for 2, 4, and 7 days. We assessed the stability of TGF-β over time at 37° C. with an enzyme-linked immunosorbent assay (ELISA) revealing slowly released from the protein substrate and its half time was 4 days. As seen in FIG. 5b, GF binding motif (ANG-MAP-RGD-IDAP) with high affinity for TGF-β exhibit long-term sustained release of TGF-β in cell culture condition.

### Examples 4. Synthetic Microenvironment to Induce Transdifferentation of Epithelial Cell to Mesenchymal Cell

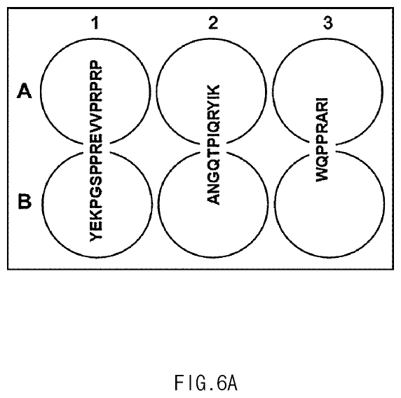

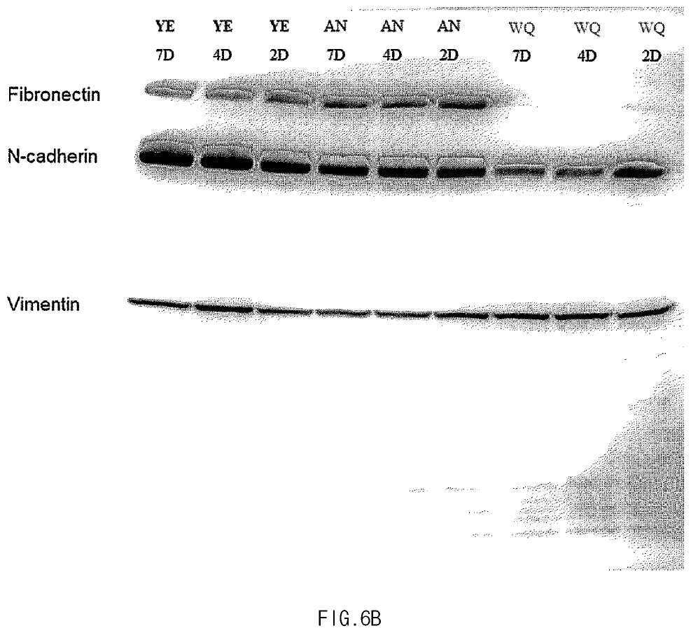

TGF-β binding motifs identified in EXAMPLES 2 and 3 were arrayed in 6 well plates. After treated with TGF-β (5 ng) as set forth in EXAMPLE 2, the 6 well plates were stored at a CO₂ incubator at 37° C. for appropriate time (e.g., 2, 4 and 7 days, respectively). MCF-10A, a breast epithelial cell, was seeded and cultured on the TGF-β binding peptide motif coated well plate. Two motifs having high affinity to TGF-β could induce MCF-10A to undergo EMT while low TGF-β binding affinity motif, WQPPRARI (SEQ ID NO: 15), could not induce EMT as evidenced by no fibronectin upregulation as seen in FIG. 6b.

### Example 5. Gf Protection from Trypsin Activity

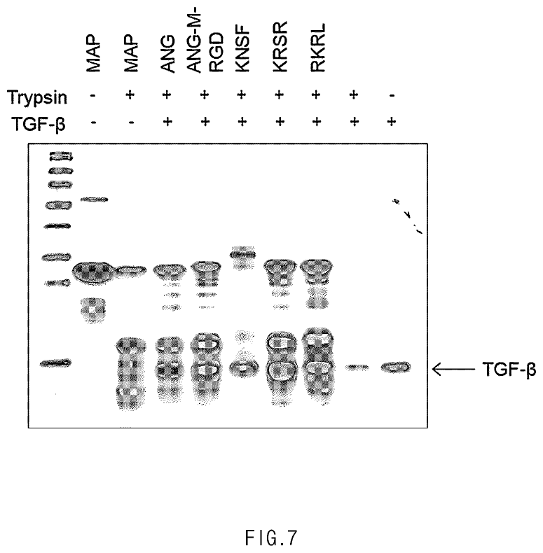

TGF-β Binding Peptide Motifs Identified in EXAMPLES 2 and 3 were incubated with TGF-β (2 μg) and trypsin (0.3 μg) at 30° C. for 15 min. After trypsinization, proteins of sample were separated by 15% SDS-PAGE in size-dependent manner. Then, SDS-PAGE was stained by Coomassie blue solution for 2 hr. After de-staining, the intensity of band, stained by Coomassie blue solution, were analyzed. ANGQTPIQRYIK (SEQ ID NO: 12), ANG-M-RGD (SEQ ID NO: 59), KNSFMALYLSKG (SEQ ID NO: 22), KRSR (SEQ ID NO: 48), and RKRLQVQLSIRT (SEQ ID NO: 20) motifs inhibited trypsinization of TGF-β compared without MAP-fusion motif as seen in FIG. 7.

### Example 6. Growth Factor Bound Protein Substrate for Cell Growth

A total of 96-well plates (non-tissue culture treated, SPL Life Science) were coated with 50 μg/ml with the protein substrate having GF binding peptide motif in sodium acetate buffer for 1 h at room temperature. 20 ng/ml of FGF2 and PDGF-BB (BioVision, Milpitas, CA, USA) in DMEM with 0.5% FBS were added to individual well and incubated for 1 hr at 37° C. in CO₂ incubator. Cell growth assays were performed using human foreskin fibroblasts (Hs68, ATCC) in DMEM medium (Invitrogen) supplemented with 0.5% fetal bovine serum (FBS). Cells were seeded at 1,500 cells/well on GF bound substrate pre-coated plates and incubated for 48 h at 37° C. in CO₂ incubator. Then, CCK-8 assay was performed to determine cell growth. Hs68 cells were checked for mycoplasma contamination and used in passages from 5 to 10. The protein substrate without GF binding peptide motif and heparin were used as negative and positive control, respectively.

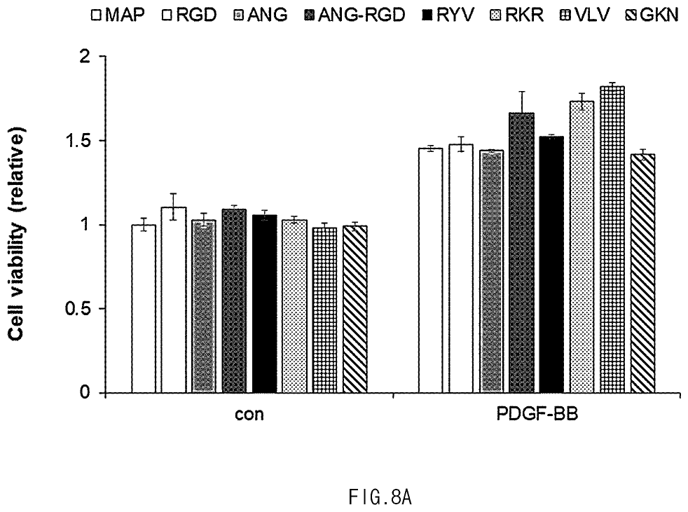

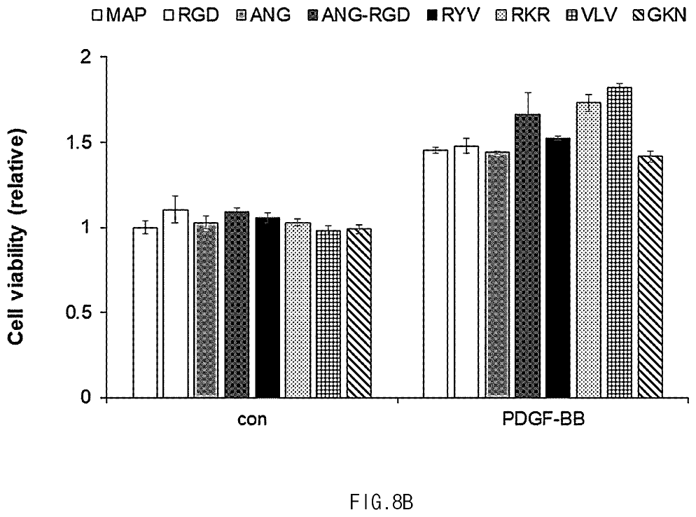

As shown in FIG. 8a and FIG. 8b, GF bound protein substrate supported cell growth and proliferation. GF bound to protein substrate strongly supported the growth and proliferation of human foreskin fibroblast in low serum condition (0.5% FBS) when compared to heparin bound GF.

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A protein substrate comprising a recombinant adhesive protein genetically functionalized with an integrin binding motif and a heparin binding motif which is capable of binding or sequestering growth factors.

2. The protein substrate of claim 1, wherein said heparin binding motif is derived from the group consisting of fibronectin domain III, laminin globular domain, heparin binding domain of collagen, vitronectin, and bone sialoprotein.

3. The protein substrate of claim 2, wherein said heparin binding motif derived from fibronectin domain III is selected from the group consisting of KYILRWRPKNS (SEQ ID NO: 7), YRVRVTPKEKTGPMKE (SEQ ID NO: 8), SPPRRARVT (SEQ ID NO: 9), ATETTITIS (SEQ ID NO: 10), VSPPRRARVTDATETTITISWRTKTETITGFG (SEQ ID NO: 11), ANGQTPIQRYIK (SEQ ID NO: 12), KPDVRSYTITG (SEQ ID NO: 13), PRARITGYIIKYEKPGSPPREVVPRPRPGV (SEQ ID NO: 14), WQPPRARI (SEQ ID NO: 15), WQPPRARITGYIIKYEKPG (SEQ ID NO: 16), YEKPGSPPREVVPRPRP (SEQ ID NO: 17), and KNNQKSEPLIGRKKT (SEQ ID NO: 18).

4. The protein substrate of claim 2, wherein said heparin binding motif derived from laminin globular domain is selected from the group consisting of GLIYYVAHQNQM (SEQ ID NO: 19), RKRLQVQLSIRT (SEQ ID NO: 20), GLLFYMARINHA (SEQ ID NO: 21), KNSFMALYLSKG (SEQ ID NO: 22), VVRDITRRGKPG (SEQ ID NO: 23), RAYFNGQSFIAS (SEQ ID NO: 24), GEKSQFSIRLKT (SEQ ID NO: 25), TLFLAHGRLVFMFNVGHKKL (SEQ ID NO: 26), TLFLAHGRLVFM (SEQ ID NO: 27), LVFMFNVGHKKL (SEQ ID NO: 28), GAAWKIKGPIYL (SEQ ID NO: 29), VIRDSNVVQLDV (SEQ ID NO: 30), GKNTGDHFVLYM (SEQ ID NO: 31), RLVSYSGVLFFLK (SEQ ID NO: 32), GPLPSYLQFVGI (SEQ ID NO: 33), RNRLHLSMLVRP (SEQ ID NO: 34), LVLFLNHGHFVA (SEQ ID NO: 35), AGQWHRVSVRWG (SEQ ID NO: 36), KMPYVSLELEMR (SEQ ID NO: 37), RYVVLPR (SEQ ID NO: 38), VRWGMQQIQLVV (SEQ ID NO: 39), TVFSVDQDNMLE (SEQ ID NO: 40), APMSGRSPSLVLK (SEQ ID NO: 41), VLVRVERATVFS (SEQ ID NO: 42), PGRWHKVSVRWE (SEQ ID NO: 76) and RNIAEIIKDI (SEQ ID NO: 43).

5. The protein substrate of claim 2, wherein said heparin binding motif derived from heparin binding domain of collagen is selected from the group consisting of KGHRGF (SEQ ID NO: 44), TAGSCLRKFSTM (SEQ ID NO: 45), and GEFYFDLRLKGDK (SEQ ID NO: 46).

6. The protein substrate of claim 2, wherein said heparin binding motif derived from heparin binding domain of vitronectin is KKQRFRHRNRKGYRSQ (SEQ ID NO: 47).

7. The protein substrate of claim 2, wherein said heparin binding motif derived from heparin binding domain of bone sialoprotein is KRSR (SEQ ID NO: 48), or KRRA (SEQ ID NO: 49).

8. The protein substrate of claim 1, wherein said heparin binding motif is capable of binding basic fibroblast growth factor (bFGF), transforming growth factor β (TGF-8), or platelet derived growth factor (PDGF).

9. The protein substrate of claim 1, wherein said integrin binding motif is selected from αvβ3-, αvβ6-, αvβ8-, α5β1-, α9β1 binding peptide.

10. The protein substrate of claim 1, wherein said integrin binding motif is capable of activating integrin αvβ6 and said heparin binding motif is capable of binding TGF-β.

11. The protein substrate of claim 1, wherein said integrin binding motif is capable of activating integrin α5β1 or α9β1 and said heparin binding motif is capable of binding bFGF.

12. The protein substrate of claim 1, wherein the recombinant adhesive protein is derived from a recombinant mussel adhesive protein.

13. The protein substrate of claim 1, wherein the recombinant mussel adhesive protein comprises the peptide selected from the group consisting of SEQ ID Nos: 1-6, and 60-74.

14. The protein substrate of claim 1, wherein the integrin binding motif and/or the heparin binding motif is bound to N-terminal and/or C-terminal of the recombinant adhesive protein.

15. The protein substrate of claim 1, wherein both of the integrin binding motif and the heparin binding motif are bound to N-terminal or C-terminal of the recombinant adhesive protein.

16. The protein substrate of claim 15, wherein the integrin binding motif and the heparin binding motif is connected via a spacer linker peptide.

17. The protein substrate of claim 16, wherein the spacer linker peptide is a peptide of SEQ ID NO: 75.

18. An extracellular microenvironment surface to regulate cell plasticity, wherein said microenvironment surface comprises the protein substrate of claim 1 that can induce combinatorial signaling via activating simultaneously integrins and growth factor receptors.

19. The extracellular microenvironment surface of claim 18, wherein said cell plasticity is epithelial-mesenchymal transition.
